(19) United States
(12) Patent Application Publication
(10) Pub. No.: US 2023/0235327 A1
(43) Pub. Date: Jul. 27, 2023

(54) A DBAIT MOLECULE IN COMBINATION WITH KRAS INHIBITOR FOR THE TREATMENT OF CANCER

(71) Applicant: ONXEO, PARIS (FR)

(72) Inventors: WAEL JDEY, MORANGIS (FR); FRANÇOISE BONO, TOULOUSE (FR)

(21) Appl. No.: 18/007,768

(22) PCT Filed: Jun. 4, 2021
(86) PCT No.: PCT/EP2021/064980

§ 371 Date: Dec. 2, 2022

(30) Foreign Application Priority Data
Jun. 5, 2020 (EP) 20305595.9

(51) Int. Cl. C12N 15/113; A61K 31/713; A61P 35/00

(52) U.S. Cl. CPC C12N 15/113; A61K 31/713; A61P 35/00; C12N 2310/531; C12N 2320/31

(57) ABSTRACT

The present invention relates to the combination of a Dbait molecule with a KRAS inhibitor for treating cancer.

### FIELD OF THE INVENTION

The present invention relates to the field of medicine, in particular of oncology.

### BACKGROUND OF THE INVENTION

RAS proteins (the four isoforms KRAS4A, KRAS4B, NRAS and HRAS encoded by three genes KRAS, NRAS and HRAS) act as molecular switches that drive several key cellular processes such as cell growth, proliferation and survival. RAS is one of the most frequently mutated oncogenes in human cancer. With about a third of all cancers driven by harmful mutations in the RAS family of genes, KRAS (Kirsten rat sarcoma 2 viral oncogene homolog) is the most frequently mutated oncogene in human tumors, causing tumor genesis and tumor maintenance. 20% of all solid tumors contain oncogenic KRAS mutations. KRAS-4B is the dominant isoform in human cancers, and it is present in approximately 90% of pancreatic cancers such as pancreatic ductal adenocarninoma (PDAC), 30% to 40% of colon cancers, and 15% to 20% of lung cancers, mostly non-small-cell lung cancer (NSCLC). It is also present in biliary tract malignancies, endometrial cancer, cervical cancer, bladder cancer, liver cancer, myeloid leukemia and breast cancer. Among the amino acid substitutions resulting from mutations, the KRAS^{G12C} mutation is the more frequent and is found in approximately 13% of people with lung cancer (˜40% in NSCLC), 3% of those with colorectal cancer, and 1% to 3% of people with other solid tumors. KRAS^{G12D} and KRAS^{G12V} mutation subtypes are believed to drive about half of all KRAS-related cancers. In colorectal cancer (CRC), KRAS^{G12V} is the most frequent, followed by KRAS^{G12D} and KRAS^{G13D}. Pancreatic ductal adenocarcinoma rather carries KRAS^{G12D} mutations (˜50-80%).

The emergence of diverse resistance mechanisms to targeted therapy is one of the foremost challenges in cancer today and limits long term efficacy of KRAS inhibitors (KRASi). Diverse drug-resistance mechanisms can arise from pre-existing mutations before treatment but more and more evidence support that small subpopulations of cancer cells can survive upon selective drug pressure. These surviving cells become Drug Tolerant Persisters (DTP), with little to-no population growth, for weeks to months, thus providing a latent reservoir of tumor cells with a “dormant” phenotype. Twenty percent of DTPs undergo phenotypic transition to become Drug Tolerant Expended Persisters which resume their proliferation, and acquire genetic modifications of resistance (e.g. EGFR T790M) at the origin of tumor recurrence in patient. Cancer therapy has traditionally focused on eliminating fast-growing populations of cells and in that case, we are face to a new paradigm. The first evidence of the role of persisters or drug tolerant cells (DTP) in targeted therapies acquired resistance mechanisms was described by Sharma et al (Cell 2010, 141, 69-80) and further described in several publications (Hata et al. Nat Med 2016, 22(3): 262-269. doi:10.1038/nm.4040., Ramirez et al. Nat Comm 2016, DOI: 10.1038/ncomms10690, Guler et al. Can Cell 2017, 32, 221-237). These works demonstrated that drug-resistance mechanisms can emerge from persisters, derived from a single, recent ancestor cell and grown under the same selective pressure. This heterogeneity presents considerable clinical challenges for ‘personalized’ therapy: even if an effective therapy is selected for one DTP, there is no guarantee that this drug would be effective for other DTPs, which in practice may have been undetected. Persisters, which are a small subpopulation of the bulk cancer population, are difficult to study in a clinical setting, and there is no known molecular signature of having passed through this state clinically. However, Hata et al provide evidence that clinically relevant drug resistant cancer cells can both pre-exist and evolve from drug tolerant cells, and point persisters as a strategic target for new therapeutic opportunities to prevent or overcome resistance in the clinic.

KRAS proteins play a major role in human cancer and have been suggested to be “undruggable” for many years. Despite three decades of intense drug discovery efforts therapies to target KRAS, no clinically feasible option for KRAS inhibition has been developed. Accordingly, new treatment methods are needed to successfully address these cells within cancer cell populations and the emergence of cancer cells resistant to therapies. Indeed, discovering new ways to eliminate the reservoir of DTPs that fail to undergo cell death, preventing mutations occurring during the transition to DTEP, is of crucial importance to cure patients.

### SUMMARY OF THE INVENTION

The Inventors of the present Invention have identified for the first time that KRAS inhibitor is associated with the occurrence of persister cancer cells during a treatment of cancer with this KRAS inhibitor. In addition, the Inventors have further surprisingly identified that the DBait molecules are capable of inhibiting the occurrence of the resistance to KRAS inhibitors and that the persister cancer cells, especially those resistant to KRAS inhibitors, are sensitive to DBait molecules (i.e., DBait is capable of leading to their cell death with a high efficiency).

The present invention provides a therapeutic agent DBait for the treatment of cancer in combination with KRAS inhibitors, in particular in order to prevent or delay the apparition of acquired resistances to the KRAS inhibitors. Indeed, the DBait molecule shows a targeted effect on persister cancer cells, thereby preventing or delaying the cancer relapse and/or preventing or delaying the apparition of acquired resistances to the KRAS inhibitors.

Accordingly, the present invention relates to a pharmaceutical composition, a combination or a kit comprising a Dbait molecule and a protein KRAS inhibitor. More specifically, the pharmaceutical composition, the combination or the kit comprises a Dbait molecule and one or several KRAS inhibitors, targeting the same or different KRAS mutant protein(s).

The present invention also relates to a pharmaceutical composition, a combination or a kit comprising a Dbait molecule and a protein KRAS inhibitor, in particular one or several KRAS inhibitors targeting the same or different KRAS mutant protein(s), for use for treating a cancer.

The present invention also relates to a Dbait molecule for use in the treatment of cancer in combination with a KRAS inhibitor, in particular one or several KRAS inhibitors targeting the same or different KRAS mutant protein(s); for use in delaying and/or preventing development of a cancer resistant to a KRAS inhibitor in a patient, in particular a KRAS inhibitor; or for use for a targeted effect against cancer persister cells in the treatment of cancer, in particular cancer persister cells to a KRAS inhibitor.

The cancer to be treated is a cancer driven by a KRAS mutation, more particularly mutation of the KRAS-4B isoform. In particular, the KRAS mutation is a KRAS^{G12C}, KRAS^{G12V}, KRAS^{G12S}, KRAS^{G12D}, KRAS^{G13C}, or KRAS^{G13D}, KRAS^{G12C}, or a KRAS^{G12D} mutation. In the context of the invention, the KRAS mutation is preferably KRAS^{G12C} mutation.

In one aspect, the KRAS inhibitor is a direct KRAS inhibitor selected from the group consisting of specific covalent KRAS inhibitors and multivalent small-molecule pan KRAS inhibitors.

The KRAS inhibitor can be selected from the group consisting of AMG-510/sotorasib (Amgen/Carmot Therapeutics), MRTX-849/Adagrasib (Mirati Therapeutics), ARS-3248/JNJ-74699157 (Johnson & Johnson/Wellspring Biosciences), Compound B (Sanofi/X-Chem Pharmaceuticals), LY3499446 (Eli Lilly), ARS-853, ARS-1620, and BI-2852, BI-1701963 (Boehringer Ingelheim), mRNA-5671 (Moderna Therapeutics), G12D inhibitor (Mirati), RAS(On)inhibitors (Revolution medicines), and BBP-454 (BridgeBio Pharma).

Optionally, the KRAS inhibitor is a KRASG12C inhibitor directly targeting and binding mutant KRASG12C protein.

Optionally, the KRAS inhibitor leaves wild-type KRAS protein untouched.

In one aspect, the Dbait molecule has at least one free end and a DNA double stranded portion of 20-200 bp with less than 60% sequence identity to any gene in a human genome. More particularly, the Dbait molecule has one of the following formulae:

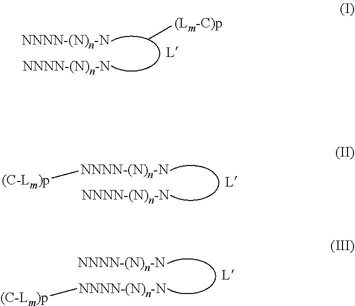

wherein N is a deoxynucleotide, n is an integer from 15 to 195, the underlined N refers to a nucleotide having or not a modified phosphodiester backbone, L′ is a linker, C is the molecule facilitating endocytosis selected from a lipophilic molecule or a ligand which targets cell receptor enabling receptor mediated endocytosis, L is a linker, m and p, independently, are an integer being 0 or 1.

Preferably, the Dbait molecule has the following formula:

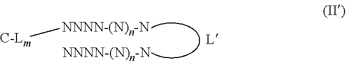

with the same definition than formulae (I), (II), and (III) for N, N, n, L, L′, C and m.

In a very specific aspect, the Dbait molecule has the following formula:

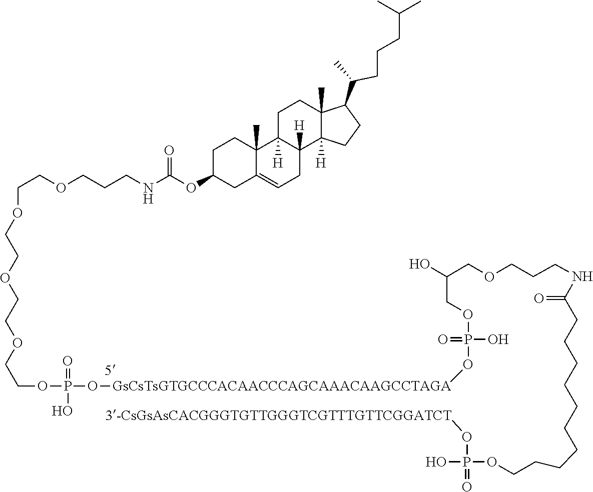

The present invention further relates to a pharmaceutical composition, a combination or the kit according to the present disclosure for use in the treatment of cancer.

It also relates to a Dbait molecule as defined herein or a pharmaceutical composition comprising it for use in the treatment of cancer in combination with a KRAS inhibitor, in particular a KRAS inhibitor as defined herein.

In addition, it relates to a Dbait molecule or a pharmaceutical composition comprising it as defined herein for use in delaying and/or preventing development of a cancer resistant to a KRAS inhibitor in a patient, in particular a KRAS inhibitor as defined herein.

In one aspect, the cancer can be selected from the group consisting of a cancer of head and neck, pancreas, stomach, colon, colorectum, small intestine, biliary tract, kidney, ovary, prostate, thyroid, esophagus, breast in particular (TNBC), bladder, lung, liver, uterine corpus, endometrium, cervix, or urinary tract, peritoneal cancers, multiple myeloma, sarcoma, skin (melanoma), in particular uveal melanoma, and hematopoietic cancers such as leukemia.

In a particular aspect, the cancer is a cancer resistant to a KRAS inhibitor.

Finally, the present invention relates to a Dbait molecule as defined herein or a pharmaceutical composition comprising it for use for a targeted effect against cancer persister cells in the treatment of cancer, in particular cancer persister cells to a KRAS inhibitor as defined herein.

### BRIEF DESCRIPTION OF THE DRAWINGS

FIG. 1. Resistance to KRAS^{G12C} evolves from Drug-Tolerant persister cells (DTCs) and AsiDNA abrogates the emergence of resistance to KRAS^{G12C} inhibitor. (A) Representative microscopy images of NCI-H23 cells, at different time points after AMG-510 (20 μM) treatment start. Senescence-like cell enlargement phenotype was analyzed at different time points after AMG-510 treatment, from day 7 to day 14. (B-C) NCI-H23 cell growth was assessed by cell counting during (B) AMG-510 (20 μM; Squares) continuous treatment alone or combined to AsiDNA (Triangles, 500 nM; Circles, 2500 nM) or (C) MRTX849 continuous treatment alone (1 μM, Triangles) or combined to AsiDNA (Squares, 2500 nM). AsiDNA was added concomitantly to AMG-510 (B) or MRTX849 (C), and continuously. (D) % β-gal positive cells were evaluated to total cell number. (E-F) Flow cytometry analysis of the kinetics of intracellular levels of p16 (E) and p21 (F) proteins in NCI-H23 cells non-treated (Parental) and AMG-510 treated. (G) Flow cytometry analysis of intracellular levels of pPERK (left) and cell-surface transferrin receptor (right) in NCI-H23 parental cells and AMG-510-induced DTCs.

FIG. 2. AsiDNA inhibits resistance to KRAS^{G12C} inhibitor even at very low doses. (A) NCI-H23 cells were treated continuously with AMG-510 alone (20 μM—Black squares) or combined to AsiDNA at different doses (ranging from 100 nM to 2500 nM). Cell growth during treatment was assessed by cell counting. (B) Sensitivity to AsiDNA of AMG-510-induced DTCs treated in (A) with AsiDNA and picked at day 14, was compared to sensitivity of NCI-H23 parental cells to AsiDNA, by cell counting. IC50s were calculated using GraphPadPrism software.

FIG. 3: KRASG12Ci-induced Drug-Tolerant persister cells are highly sensitive to AsiDNA. (A—left part) NCI-H23 cells were treated continuously with AMG-510 (20 μM) and (A—right part) sensitivity of DTCs (Squares) and parental cells (Circles) to increasing doses of AMG-510 or AsiDNA was assessed 1 week after treatment by XTT assay. IC₅₀ were calculated using GraphPadPrism software. (B) Representative microscopy images of γH2AX staining in NCI-H23 parental or in NCI-H23 DTCs treated during 24 hours with AsiDNA (100 nM and 5000 nM). DAPI,grey; γH2AX,white.

FIG. 4: AsiDNA prevents resistance to KRAS^{G12C}i in pancreatic cancer. (A) MIA PaCa-2 cells were treated continuously with AMG-510 (1 μM—left part) or MRTX-849 (1 μM—right part) with AsiDNA (Grey Circles) or without AsiDNA (Black Squares) and cell survival assessed every week by cell counting. (B) Sensitivity of DTCs (Grey Squares) and parental cells (Black Circles) to increasing doses of AsiDNA™ was assessed 1 week after treatment by XTT assay. IC₅₀ were calculated using GraphPadPrism software.

### DETAILED DESCRIPTION OF THE INVENTION

The present invention relates to the capacity of a Dbait molecule to strongly decrease the emergence of proliferative cells from persistent cancer cells.

Accordingly, the present invention relates to a pharmaceutical composition, a combination or a kit (kit-of-parts) comprising a Dbait molecule and a KRAS inhibitor, in particular for use for treating cancer. More specifically, the pharmaceutical composition, the combination or the kit comprises a Dbait molecule and one or several KRAS inhibitors, targeting the same or different KRAS(s).

The present invention also relates to a pharmaceutical composition comprising a Dbait molecule and a KRAS inhibitor for use in the treatment of a cancer; to a combination or a kit (kit-of-parts) comprising a Dbait molecule and a KRAS inhibitor as a combined preparation for simultaneous, separate or sequential use, in particular for use in the treatment of cancer. It further relates to a method for treating a cancer in a subject in need thereof, comprising administering a therapeutically effective amount of a Dbait molecule and a therapeutically effective amount of a KRAS inhibitor, and optionally a pharmaceutically acceptable carrier. It relates to the use of a Dbait molecule and a KRAS inhibitor for the manufacture of a drug for treating a cancer. It also relates to the use of a pharmaceutical composition, a combination or a kit according to the present disclosure for the manufacture of a medicament for the treatment of cancer.

The present invention relates to a Dbait molecule or a pharmaceutical composition comprising a Dbait molecule for use for the treatment of cancer in combination with a KRAS inhibitor. More particularly, it relates to a Dbait molecule or a pharmaceutical composition comprising a Dbait molecule for use in delaying and/or preventing development of a cancer resistant to a KRAS inhibitor in a patient. It relates to a Dbait molecule for use in extending the duration of response to a KRAS inhibitor in the cancer treatment of a patient. It also relates to a method for delaying and/or preventing development of a cancer resistant to a KRAS inhibitor in a patient and/or for extending the duration of response to a KRAS inhibitor in the cancer treatment of a patient, comprising administering a therapeutically effective amount of a Dbait molecule and a therapeutically effective amount of a KRAS inhibitor, and optionally a pharmaceutically acceptable carrier. It relates to the use of a Dbait molecule for the manufacture of a drug for treating a cancer in combination with a KRAS inhibitor, for delaying and/or preventing development of a cancer resistant to a KRAS inhibitor in a patient and/or for extending the duration of response to a KRAS inhibitor in the cancer treatment of a patient. It relates to the use of a Dbait molecule as defined herein or a pharmaceutical composition comprising it for the manufacture of a medicament for the treatment of cancer in combination with a KRAS inhibitor. It relates to the use of a Dbait molecule as defined herein or a pharmaceutical composition comprising it for the manufacture of a medicament for delaying and/or preventing development of a cancer resistant to a KRAS inhibitor in a patient, in particular a KRAS inhibitor as defined herein.

Finally, more generally, the present invention relates to a Dbait molecule for use for inhibiting or preventing proliferation of cancer cells from persistent cells by inducing persistent cells death thereby preventing or delaying the cancer relapse and/and the emergence of acquired resistance to a cancer treatment, in particular to a KRAS inhibitor treatment. In addition, this effect against cancer persistent cells may allow to reach a complete response to the cancer treatment. Indeed, the Dbait molecule would be able to eliminate the cancer persistent cells. It also relates to a method for removing or decreasing the cancer persister cell population and/or for preventing or delaying the cancer relapse and/or the emergence of acquired resistance to a cancer treatment, in particular to a KRAS inhibitor treatment, comprising administering a therapeutically effective amount of a Dbait molecule, thereby removing or decreasing the cancer persister cell population. It relates to the use of a Dbait molecule as defined herein or a pharmaceutical composition comprising it for the manufacture of a medicament for a targeted effect against cancer persister cells in the treatment of cancer, in particular cancer persister cells to a KRAS inhibitor as defined herein. The Dbait treatment would be beneficial in targeting viable “persister” tumor cells and thus may prevent the emergence of drug-resistant clone(s), in particular in the context of a combined treatment with a KRAS inhibitor.

### Definition

The terms “kit”, “product”, “combination” or “combined preparation”, as used herein, defines especially a “kit-of-parts” in the sense that the combination partners as defined above can be dosed independently or by use of different fixed combinations with distinguished amounts of the combination partners, i.e. simultaneously or at different time points. The parts of the kit-of-parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

Within the context of the invention, the term “treatment” denotes curative, symptomatic, preventive treatment as well as maintenance treatment. Pharmaceutical compositions, kits, products and combined preparations of the invention can be used in humans with existing cancer or tumor, including at early or late stages of progression of the cancer. The pharmaceutical compositions, kits, combinations, products and combined preparations of the invention will not necessarily cure the patient who has the cancer but will delay or slow the progression or prevent further progression of the disease, ameliorating thereby the patients' condition. In particular, the pharmaceutical compositions, kits, combinations, products and combined preparations of the invention reduce the development of tumors, reduce tumor burden, produce tumor regression in a mammalian host and/or prevent metastasis occurrence and cancer relapse. The pharmaceutical compositions, kits, combinations, products and combined preparations according to the present invention advantageously prevent, delay the emergence or the development of, decrease or remove the persister tumor cells and/or drug-tolerant expanded persisters.

By “therapeutically effective amount” it is meant the quantity of the compound of interest of the pharmaceutical composition, kit, combination, product or combined preparation of the invention which prevents, removes or reduces the deleterious effects of cancer in mammals, including humans, alone or in combination with the other active ingredients of the pharmaceutical composition, kit, combination, product or combined preparation. It is understood that the administered dose may be lower for each compound in the composition to the “therapeutically effective amount” define for each compound used alone or in combination with other treatments than the combination described here. The “therapeutically effective amount” of the composition will be adapted by those skilled in the art according to the patient, the pathology, the mode of administration, etc.

Whenever within this whole specification the terms “treatment of a cancer” or “treating a cancer” or the like are mentioned with reference to the pharmaceutical composition, kit, combination, product or combined preparation of the invention, there is meant: a) a method for treating a cancer, said method comprising administering a pharmaceutical composition, kit, combination, product or combined preparation of the invention to a patient in need of such treatment; b) the use of a pharmaceutical composition, kit, combination, product or combined preparation of the invention for the treatment of a cancer; c) the use of a pharmaceutical composition, kit, combination, product or combined preparation of the invention for the manufacture of a medicament for the treatment of a cancer; and/or d) a pharmaceutical composition, kit, combination, product or combined preparation of the invention for use in the treatment a cancer.

The pharmaceutical compositions, kits, combinations, products or combined preparations contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition, kit, combination, product or combined preparation can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient(s); in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavouring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions, kits, combinations, products or combined preparations are advantageously applied by injection or intravenous infusion of suitable sterile solutions or as oral dosage by the digestive tract. Methods for the safe and effective administration of most of these therapeutic agents are known to those skilled in the art. In addition, their administration is described in the standard literature.

“KRAS” or “GTPase KRas” refers to a protein as defined in UniProt under accession number P01116 and in NCBI Reference Sequence NP_203524.1.

The term “KRAS inhibitor” or “K-RAS inhibitor” means a compound that inhibits or reduces the activity or level (e.g. amount) of a signaling pathway of one or more KRAS protein(s) (KRAS4A, K-RAS4B), and preferably of mutant KRAS protein(s) (KRAS^{G12C}, KRAS^{G12V}, KRAS^{G12S}, KRAS^{G12D}, KRAS^{G13C}, KRAS^{G13D}).

By “persister cell”, “persister cancer cell”, “drug tolerant persister” or “DTP” is intended to refer to a small subpopulation of cancer cells that maintain viability under anti-cancer targeted therapy treatments, in particular a treatment with a KRAS inhibitor. More particularly, it refers to cancer cells that have a tolerance to high concentrations of a treatment of a KRAS inhibitor, when it is used in concentrations that are 100 of times higher than IC50. These cells have a slow growth and are almost quiescent.

The term “drug-tolerant expanded persister” or “DTEP” as used herein, refers to cancer cells that are capable to survive with continuous cancer drug treatment in high concentrations, in particular a treatment with a KRAS inhibitor.

### Dbait Molecules

The term “Dbait molecule” also known as signal interfering DNA (siDNA) as used herein, refers to a nucleic acid molecule, preferably a hairpin nucleic acid molecule, designed to counteract DNA repair. A Dbait molecule has at least one free end and a DNA double stranded portion of 20-200 bp with less than 60% sequence identity to any gene in a human genome.

Preferably, the Dbait molecules for use in the present invention, conjugated or not, can be described by the following formulae:

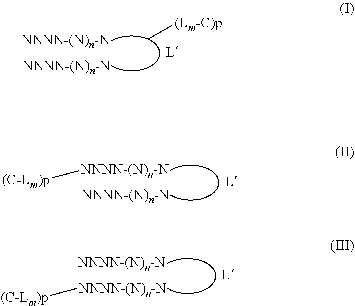

wherein N is a deoxynucleotide, n is an integer from 15 to 195, the underlined N refers to a nucleotide having or not a modified phosphodiester backbone, L′ is a linker, C is a molecule facilitating endocytosis preferably selected from a lipophilic molecule and a ligand which targets cell receptor enabling receptor mediated endocytosis, L is a linker, m and p, independently, are an integer being 0 or 1.

In preferred embodiments, the Dbait molecules of formulae (I), (II), or (III) have one or several of the following features:
N is a deoxynucleotide, preferably selected from the group consisting of A (adenine), C (cytosine), T (thymine) and G (guanine) and selected so as to avoid occurrence of a CpG dinucleotide and to have less than 80% or 70%, even less than 60% or 50% sequence identity to any gene in a human genome; and/or,
n is an integer from 15 to 195, from 19-95, from 21 to 95, from 27 to 95, from 15 to 45, from 19 to 45, from 21 to 45, or from 27 to 45; preferably n is 27; and/or,
the underlined N refers to a nucleotide having or not a phosphorothioate or methylphosphonate backbone, more preferably a phosphorothioate backbone; preferably, the underlined N refers to a nucleotide having a modified phosphodiester backbone; and/or,
the linker L′ is selected from the group consisting of hexaethyleneglycol, tetradeoxythymidylate (T4), 1,19-bis(phospho)-8-hydraza-2-hydroxy-4-oxa-9-oxo-nonadecane; and 2,19-bis(phosphor)-8-hydraza-1-hydroxy-4-oxa-9-oxo-nonadecane; and/or,
m is 1 and L is a carboxamido polyethylene glycol, more preferably carboxamido triethylene or tetraethylene glycol; and/or,
C is selected from the group consisting of a cholesterol, single or double chain fatty acids such as octadecyl, oleic acid, dioleoyl or stearic acid, or ligand (including peptide, protein, aptamer) which targets cell receptor such as folic acid, tocopherol, sugar such as galactose and mannose and their oligosaccharide, peptide such as RGD and bombesin, and protein such transferring and integrin, preferably is a cholesterol or a tocopherol, still more preferably a cholesterol.

Preferably, C-Lm is a triethyleneglycol linker (10-O-[1-propyl-3-N-carbamoylcholesteryl]-triethyleneglycol radical. Alternatively, C-Lm is a tetraethyleneglycol linker (13-O-[1-propyl-3-N-carbamoylcholesteryl]-tetraethyleneglycol radical.

In a preferred embodiment, the Dbait molecule has the following formula:

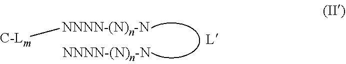

with the same definition than formulae (I), (II), and (III) for N, N, n, L, L′, C and m.

In a particular embodiment, the Dbait molecules are those extensively described in PCT patent applications WO2005/040378, WO2008/034866, WO2008/084087 and WO2011/161075, the disclosure of which is incorporated herein by reference.

Dbait molecules may be defined by a number of characteristics necessary for their therapeutic activity, such as their minimal length, the presence of at least one free end, and the presence of a double stranded portion, preferably a DNA double stranded portion. As will be discussed below, it is important to note that the precise nucleotide sequence of Dbait molecules does not impact on their activity. Furthermore, Dbait molecules may contain a modified and/or non-natural backbone.

Preferably, Dbait molecules are of non-human origin (i.e., their nucleotide sequence and/or conformation (e.g., hairpin) does not exist as such in a human cell), most preferably of synthetic origin. As the sequence of the Dbait molecules plays little, if any, role, Dbait molecules have preferably no significant degree of sequence homology or identity to known genes, promoters, enhancers, 5′- or 3′-upstream sequences, exons, introns, and the like. In other words, Dbait molecules have less than 80% or 70%, even less than 60% or 50% sequence identity to any gene in a human genome. Methods of determining sequence identity are well known in the art and include, e.g., Blast. Dbait molecules do not hybridize, under stringent conditions, with human genomic DNA. Typical stringent conditions are such that they allow the discrimination of fully complementary nucleic acids from partially complementary nucleic acids.

In addition, the sequence of the Dbait molecules is preferably devoid of CpG in order to avoid the well-known toll-like receptor-mediated immunological reactions.

The length of Dbait molecules may be variable, as long as it is sufficient to allow appropriate binding of Ku protein complex comprising Ku and DNA-PKcs proteins. It has been showed that the length of Dbait molecules must be greater than 20 bp, preferably about 32 bp, to ensure binding to such a Ku complex and allowing DNA-PKcs activation. Preferably, Dbait molecules comprise between 20-200 bp, more preferably 24-100 bp, still more preferably 26-100, and most preferably between 24-200, 25-200, 26-200, 27-200, 28-200, 30-200, 32-200, 24-100, 25-100, 26-100, 27-100, 28-100, 30-100, 32-200 or 32-100 bp. For instance, Dbait molecules comprise between 24-160, 26-150, 28-140, 28-200, 30-120, 32-200 or 32-100 bp. By “bp” is intended that the molecule comprise a double stranded portion of the indicated length.

In a particular embodiment, the Dbait molecules having a double stranded portion of at least 32 pb, or of about 32 bp, comprise the same nucleotide sequence than Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5). Optionally, the Dbait molecules have the same nucleotide composition than Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) but their nucleotide sequence is different. Then, the Dbait molecules comprise one strand of the double stranded portion with 3 A, 6 C, 12 G and 11 T. Preferably, the sequence of the Dbait molecules does not contain any CpG dinucleotide.

Alternatively, the double stranded portion comprises at least 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5). In a more particular embodiment, the double stranded portion consists in 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5).

The Dbait molecules as disclosed herein must have at least one free end, as a mimic of double strand breaks (DSB). Said free end may be either a free blunt end or a 573T-protruding end. The “free end” refers herein to a nucleic acid molecule, in particular a double-stranded nucleic acid portion, having both a 5′ end and a 3′ end or having either a 3′end or a 5′ end. Optionally, one of the 5′ and 3′ end can be used to conjugate the nucleic acid molecule or can be linked to a blocking group, for instance a or 3′-3′nucleotide linkage.

In a particular embodiment, they contain only one free end. Preferably, Dbait molecules are made of hairpin nucleic acids with a double-stranded DNA stem and a loop. The loop can be a nucleic acid, or other chemical groups known by skilled person or a mixture thereof. A nucleotide linker may include from 2 to 10 nucleotides, preferably, 3, 4 or 5 nucleotides. Non-nucleotide linkers non-exhaustively include abasic nucleotide, polyether, polyamine, polyamide, peptide, carbohydrate, lipid, polyhydrocarbon, or other polymeric compounds (e.g. oligoethylene glycols such as those having between 2 and 10 ethylene glycol units, preferably 3, 4, 5, 6, 7 or 8 ethylene glycol units). A preferred linker is selected from the group consisting of hexaethyleneglycol, tetradeoxythymidylate (T4) and other linkers such as 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane. Accordingly, in a particular embodiment, the Dbait molecules can be a hairpin molecule having a double stranded portion or stem comprising at least 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) and a loop being a hexaethyleneglycol linker, a tetradeoxythymidylate linker (T4) 1,19-bis(phospho)-8-hydraza-2-hydroxy-4-oxa-9-oxo-nonadecane or 2,19-bis(phosphor)-8-hydraza-1-hydroxy-4-oxa-9-oxo-nonadecane. In a more particular embodiment, those Dbait molecules can have a double stranded portion consisting in 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5).

Dbait molecules preferably comprise a 2′-deoxynucleotide backbone, and optionally comprise one or several (2, 3, 4, 5 or 6) modified nucleotides and/or nucleobases other than adenine, cytosine, guanine and thymine. Accordingly, the Dbait molecules are essentially a DNA structure. In particular, the double-strand portion or stem of the Dbait molecules is made of deoxyribonucleotides.

Preferred Dbait molecules comprise one or several chemically modified nucleotide(s) or group(s) at the end of one or of each strand, in particular in order to protect them from degradation. In a particular preferred embodiment, the free end(s) of the Dbait molecules is(are) protected by one, two or three modified phosphodiester backbones at the end of one or of each strand. Preferred chemical groups, in particular the modified phosphodiester backbone, comprise phosphorothioates. Alternatively, preferred Dbait have 3′-3′ nucleotide linkage, or nucleotides with methylphosphonate backbone. Other modified backbones are well known in the art and comprise phosphoramidates, morpholino nucleic acid, 2′-0,4′-C methylene/ethylene bridged locked nucleic acid, peptide nucleic acid (PNA), and short chain alkyl, or cycloalkyl intersugar linkages or short chain heteroatomic or heterocyclic intrasugar linkages of variable length, or any modified nucleotides known by skilled person. In a first preferred embodiment, the Dbait molecules have the free end(s) protected by one, two or three modified phosphodiester backbones at the end of one or of each strand, more preferably by three modified phosphodiester backbones (in particular phosphorothioate or methylphosphonate) at least at the 3′end, but still more preferably at both 5′ and 3′ ends.

In a most preferred embodiment, the Dbait molecule is a hairpin nucleic acid molecule comprising a DNA double-stranded portion or stem of 32 bp (e.g., with a sequence selected from the group consisting of SEQ ID Nos 1-5, in particular SEQ ID No 4) and a loop linking the two strands of the DNA double-stranded portion or stem comprising or consisting of a linker selected from the group consisting of hexaethyleneglycol, tetradeoxythymidylate (T4) and 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane, the free ends of the DNA double-stranded portion or stem (i.e. at the opposite of the loop) having three modified phosphodiester backbones (in particular phosphorothioate internucleotidic links).

Said nucleic acid molecules are made by chemical synthesis, semi-biosynthesis or biosynthesis, any method of amplification, followed by any extraction and preparation methods and any chemical modification. Linkers are provided so as to be incorporable by standard nucleic acid chemical synthesis. More preferably, nucleic acid molecules are manufactured by specially designed convergent synthesis: two complementary strands are prepared by standard nucleic acid chemical synthesis with the incorporation of appropriate linker precursor, after their purification, they are covalently coupled together.

Optionally, the nucleic acid molecules may be conjugated to molecules facilitating endocytosis or cellular uptake.

In particular, the molecules facilitating endocytosis or cellular uptake may be lipophilic molecules such as cholesterol, single or double chain fatty acids, or ligands which target cell receptor enabling receptor mediated endocytosis, such as folic acid and folate derivatives or transferrin (Goldstein et al. Ann. Rev. Cell Biol. 1985 1:1-39; Leamon & Lowe, Proc Natl Acad Sci USA. 1991, 88: 5572-5576.). The molecule may also be tocopherol, sugar such as galactose and mannose and their oligosaccharide, peptide such as RGD and bombesin and protein such as integrin. Fatty acids may be saturated or unsaturated and be in C₄-C₂₈, preferably in C₁₄-C₂₂, still more preferably being in C₁₈ such as oleic acid or stearic acid. In particular, fatty acids may be octadecyl or dioleoyl. Fatty acids may be found as double chain form linked with in appropriate linker such as a glycerol, a phosphatidylcholine or ethanolamine and the like or linked together by the linkers used to attach on the Dbait molecule. As used herein, the term “folate” is meant to refer to folate and folate derivatives, including pteroic acid derivatives and analogs. The analogs and derivatives of folic acid suitable for use in the present invention include, but are not limited to, antifolates, dihydrofolates, tetrahydrofolates, folinic acid, pteropolyglutamic acid, 1-deza, 3-deaza, 5-deaza, 8-deaza, 10-deaza, 1,5-deaza, 5,10 dideaza, 8,10-dideaza, and 5,8-dideaza folates, antifolates, and pteroic acid derivatives. Additional folate analogs are described in U52004/242582. Accordingly, the molecule facilitating endocytosis may be selected from the group consisting of single or double chain fatty acids, folates and cholesterol. More preferably, the molecule facilitating endocytosis is selected from the group consisting of dioleoyl, octadecyl, folic acid, and cholesterol. In a most preferred embodiment, the nucleic acid molecule is conjugated to a cholesterol.

The Dbait molecules facilitating endocytosis may be conjugated to molecules facilitating endocytosis, preferably through a linker. Any linker known in the art may be used to attach the molecule facilitating endocytosis to Dbait molecules. For instance, WO09/126933 provides a broad review of convenient linkers pages 38-45. The linker can be non-exhaustively, aliphatic chain, polyether, polyamine, polyamide, peptide, carbohydrate, lipid, polyhydrocarbon, or other polymeric compounds (e.g. oligoethylene glycols such as those having between 2 and 10 ethylene glycol units, preferably 3, 4, 5, 6, 7 or 8 ethylene glycol units, still more preferably 3 ethylene glycol units), as well as incorporating any bonds that may be break down by chemical or enzymatical way, such as a disulfide linkage, a protected disulfide linkage, an acid labile linkage (e.g., hydrazone linkage), an ester linkage, an ortho ester linkage, a phosphonamide linkage, a biocleavable peptide linkage, an azo linkage or an aldehyde linkage. Such cleavable linkers are detailed in WO2007/040469 pages 12-14, in WO2008/022309 pages 22-28.

In a particular embodiment, the nucleic acid molecule can be linked to one molecule facilitating endocytosis. Alternatively, several molecules facilitating endocytosis (e.g., two, three or four) can be attached to one nucleic acid molecule.

In a specific embodiment, the linker between the molecule facilitating endocytosis, in particular cholesterol, and nucleic acid molecule is CO—NH—CH₂—(CH₂—CH₂—O)ₙ, wherein n is an integer from 1 to 10, preferably n being selected from the group consisting of 3, 4, 5 and 6. In a very particular embodiment, the linker is CO—NH—CH₂—(CH₂—CH₂—O)₄ (carboxamido tetraethylene glycol) or CO—NH—CH₂—(CH₂—CH₂—O)₃ (carboxamido triethylene glycol). The linker can be linked to nucleic acid molecules at any convenient position which does not modify the activity of the nucleic acid molecules. In particular, the linker can be linked at the 5′ end. Therefore, in a preferred embodiment, the contemplated conjugated Dbait molecule is a Dbait molecule having a hairpin structure and being conjugated to the molecule facilitating endocytosis, preferably through a linker, at its 5′ end.

In another specific embodiment, the linker between the molecule facilitating endocytosis, in particular cholesterol, and nucleic acid molecule is dialkyl-disulfide {e.g., (CH₂)ᵣ—S—S—(CH₂)ₛ with r and s being integer from 1 to 10, preferably from 3 to 8, for instance 6}.

In a most preferred embodiment, the conjugated Dbait molecule is a hairpin nucleic acid molecule comprising a DNA double-stranded portion or stem of 32 bp and a loop linking the two strands of the DNA double-stranded portion or stem comprising or consisting of a linker selected from the group consisting of hexaethyleneglycol, tetradeoxythymidylate (T4), 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane, the free ends of the DNA double-stranded portion or stem (i.e. at the opposite of the loop) having three modified phosphodiester backbones (in particular phosphorothioate internucleotidic links) and said Dbait molecule being conjugated to a cholesterol at its 5′ end, preferably through a linker (e.g. carboxamido oligoethylene glycol, preferably carboxamido triethylene or tetraethylene glycol).

In a particular embodiment, the Dbait molecules can be conjugated Dbait molecules such as those extensively described in PCT patent application WO2011/161075, the disclosure of which is incorporated herein by reference.

In a preferred embodiment, NNNN—(N)ₙ—N comprises at least 6, 8, 10, 12, 14, 16, 18, 20, 22, 24, 26, 28, 30 or 32 consecutive nucleotides of Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5) or consists in 20, 22, 24, 26, 28, 30 or 32 consecutive nucleotides of Dbait32, Dbait32Ha, Dbait32Hb, Dbait32Hc or Dbait32Hd. In a particular embodiment, NNNN—(N)ₙ—N comprises or consists in Dbait32 (SEQ ID NO: 1), Dbait32Ha (SEQ ID NO: 2), Dbait32Hb (SEQ ID NO: 3), Dbait32Hc (SEQ ID NO: 4) or Dbait32Hd (SEQ ID NO: 5), more preferably Dbait32Hc (SEQ ID NO: 4).

According, the conjugated Dbait molecules may be selected from the group consisting of:

with NNNN—(N)ₙ—N being SEQ ID NO: 1;
with NNNN—(N)ₙ—N being SEQ ID NO: 2;
with NNNN—(N)ₙ—N being SEQ ID NO: 3;
with NNNN—(N)ₙ—N being SEQ ID NO: 4; or
with NNNN—(N)ₙ—N being SEQ ID NO: 5

In one preferred embodiment, the Dbait molecule has the following formula:

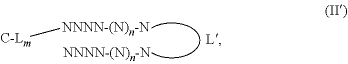

wherein
NNNN—(N)ₙ—N comprises 28, 30 or 32 nucleotides, preferably 32 nucleotides; and/or
the underlined nucleotide refers to a nucleotide having or not a phosphorothioate or methylphosphonate backbone, more preferably a phosphorothioate backbone; preferably, the underlined nucleotide refers to a nucleotide having a phosphorothioate or methylphosphonate backbone, more preferably a phosphorothioate backbone; and/or,
the linker L′ is selected from the group consisting of hexaethyleneglycol, tetradeoxythymidylate (T4), 1,19-bis(phospho)-8-hydraza-2-hydroxy-4-oxa-9-oxo-nonadecane or 2,19-bis(phosphor)-8-hydraza-1-hydroxy-4-oxa-9-oxo-nonadecane; and/or,
m is 1 and L is a carboxamido polyethylene glycol, more preferably carboxamido triethylene or tetraethylene glycol; and/or,
C is selected from the group consisting of a cholesterol, single or double chain fatty acids such as octadecyl, oleic acid, dioleoyl or stearic acid, or ligand (including peptide, protein, aptamer) which targets cell receptor such as folic acid, tocopherol, sugar such as galactose and mannose and their oligosaccharide, peptide such as RGD and bombesin, and protein such transferring and integrin, preferably is a cholesterol.

In a very specific embodiment, the Dbait molecule (also referred herein as AsiDNA) has the following formula:

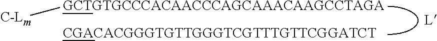

(IIa) (SEQ ID NO: 6) wherein C is a cholesteryl, Lm is a carboxamido tetraethylene glycol, and L′ is 1,19-bis(phospho)-8-hydraza-2-hydroxy-4-oxa-9-oxo-nonadecane; also represented by the following formula:

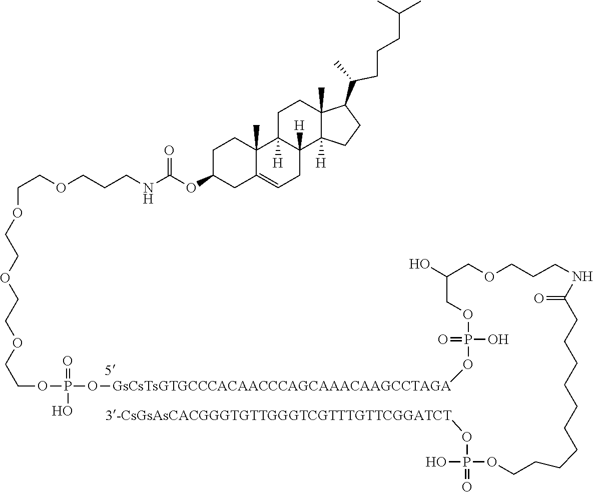

“s” refers to a phosphorothioate link between two nucleotides.

### KRAS Inhibitors

The KRAS inhibitor of the present invention is a KRAS inhibitor for treating cancer, preferably a cancer driven by a KRAS mutation. In particular, the KRAS mutation is selected from a KRAS^{G12C}, KRAS^{G12V}, KRAS^{G12S}, KRAS^{G12D}, KRAS^{G13C}, or KRAS^{G13D}, KRAS^{G12C}, or KRAS^{G12D} mutation. In the context of the invention, the KRAS mutation is preferably a KRAS^{G12C} mutation.

In a particular aspect, the KRAS inhibitor is known to be associated with an acquired resistance during the cancer treatment. In a very particular aspect, the Inventors of the present invention have identified for the first time that KRAS inhibitor is associated with the occurrence of persister cancer cells during a treatment of cancer with this KRAS inhibitor.

The KRAS inhibitor directly or indirectly inhibits the mutant KRAS protein. The KRAS inhibitor inhibits, prevents or reduces the activity or level (e.g. amount) of a signaling pathway of one or more KRAS protein(s), and preferably of mutant KRAS proteins.

In one aspect, the KRAS inhibitor directly targets the mutant KRAS, for instance by covalently targeting and binding mutant KRAS. In another aspect, the KRAS inhibitor indirectly targets the mutant KRAS, acts against the crucial steps required for KRAS activation, for instance by targeting and inhibiting KRAS interaction with associated proteins required for membrane association, by inhibiting KRAS-driven malignant phenotypes and or via KRAS synthetic lethal interactions.

The KRAS inhibitor can target the same KRAS mutant protein (for instance the KRAS inhibitor only targets KRAS^{G12C} mutant protein) or different KRAS mutant proteins (for instance the KRAS inhibitor targets several KRAS^{G12C}, KRAS^{G12D} and KRAS^{G13C} mutant proteins).

In a one preferred aspect, the KRAS inhibitor is a KRAS inhibitor that selectively target one or several mutant protein(s) and leaves wild-type KRAS protein untouched.

In a preferred aspect, the KRAS inhibitor is a direct KRAS inhibitor selected from the group consisting of specific covalent KRAS inhibitors (electrophilic KRAS inhibitors forming irreversible covalent bonds) and multivalent small-molecule pan KRAS inhibitor.

In one preferably aspect, direct KRAS inhibitor is a KRAS inhibitor directly targeting and binding mutant KRAS protein selected from the group consisting of AMG-510/Sotorasib (Amgen/Carmot Therapeutics, CAS NUMBER 2252403-56-6, 4-((S)-4-acryloyl methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one), MRTX-849/Adagrasib (Mirati Therapeutics, CAS NUMBER 2326521-71-3, 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoroacryloyl)piperazin-2-yl)acetonitrile), ARS-3248/JNJ-74699157 (Johnson & Johnson/Wellspring Biosciences), Compound B (Sanofi/X-Chem Pharmaceuticals), LY3499446 (Eli Lilly), ARS-853, ARS-1620, BI-2852, BI-1701963 (Boehringer Ingelheim), mRNA-5671 (Moderna Therapeutics), G12D inhibitor (Mirati), RAS(On)inhibitors (Revolution medicines), and BBP-454 (BridgeBio Pharma). In one preferably aspect, direct KRAS inhibitor is a KRAS^{G12C} inhibitor directly targeting and binding mutant KRAS^{G12C} and forming irreversible covalent bonds with nucleophilic sulfur atom of Cys-12 (the amino acid at position 12 in KRAS with the G12C mutation is a cysteine instead of glycine).

In a particular embodiment, the KRAS^{G12C} inhibitor selectively targets mutant KRAS protein and leaves wild-type KRAS untouched.

The direct KRAS inhibitor is a small organic molecule. The term excludes biological macromolecules (e.g. proteins, nucleic acids, etc.). Preferred small organic molecules range in size up to 2000 Da, and most preferably up to about 1000 Da.

Example of KRAS inhibitors can be found in the following non-exhaustive list of patent applications, WO2017058805, WO2016172692, WO2015054572, WO2017058902, WO2017058728, WO2017058807, WO2016172187, WO2018011351, WO2016164675, WO2018119183, WO2016049524, WO2017058792, WO2016168540, WO2014152588, WO2017015562, WO2018064510, WO2017172979, WO2019110751, WO2018140514, WO2019055540, WO2018068017, WO2018206539, WO2018195439, WO2015179434, WO2016179558, WO2018140600, the disclosure thereof being incorporated herein by reference; or in the following reviews, the disclosure thereof being incorporated herein by reference: Nagasaka et al, Cancer Treat Rev. 2020 March; 84:10197; Khan et al, Biochim Biophys Acta Mol Cell Res. 2020 February; 1867(2):118570; Liu et al, Acta Pharm Sin B. 2019 September; 9(5):871-879, Wu et al Curr Top Med Chem. 2019; 19(23):2081-2097.

In another aspect of the invention, indirect KRAS therapeutic strategies towards KRAS-driven cancer can be used in combination with the Dbait according to the invention.

In one aspect, indirect KRAS therapy can be selected from KRAS therapy targeting KRAS signaling pathway, for instance targeting KRAS directed to membrane association for example with farnesyltransferase inhibitor (FTI), geranylgeranyltransferase inhibitor (GGTI), prenyl-binding protein (PDEδ) inhibitor such as Deltarasin or Deltazinone 1 interfering with binding of PDEδ to KRAS and impairs KRAS localization to endomembrane; or KRAS therapy exploiting KRAS-regulated metabolic pathways for example by inhibiting glyceraldehyde 3-phosphate dehydrogenase (GAPDH); or SHP2 lethality approach (synthetic lethality interactor with KRAS oncogene) targeting various mutations including KRAS^{G12C}, for instance SHP099 or RMC-4550; or anti-KRAS immunotherapy for example with antibodies (neutralizing monoclonal antibodies), NK cell-based cancer immunotherapy involving FBP1 targeting, or engineered T-cell receptor (for instance anti-KRASG12D engineered T-cell receptor); or mRNA KRAS vaccine strategy.

### Additional Therapies

Optionally, the treatment with a nucleic acid molecule as disclosed herein and a KRAS inhibitor can be used in combination with a radiotherapy, a radioisotope therapy and/or another antitumor chemotherapy, immunotherapy, or hormonal therapy. Preferably, the antitumor chemotherapy is a treatment by a DNA damaging antitumor agent, either directly or indirectly.

As used herein, the term “antitumor chemotherapy” or “chemotherapy” refers to a cancer therapeutic treatment using chemical or biochemical substances, in particular using one or several antineoplastic agents. In particular, it also includes hormonal therapy and immunotherapy. The term “hormonal therapy” refers to a cancer treatment having for purpose to block, add or remove hormones. For instance, in breast cancer, the female hormones estrogen and progesterone can promote the growth of some breast cancer cells. So in these patients, hormone therapy is given to block estrogen and a non-exhaustive list commonly used drugs includes: Tamoxifen, Fareston, Arimidex, Aromasin, Femara, Zoladex/Lupron, Megace, and Halotestin. The term “immunotherapy” refers to a cancer therapeutic treatment using the immune system to reject cancer. The therapeutic treatment stimulates the patient's immune system to attack the malignant tumor cells.

In a particular aspect, the nucleic acid molecule as disclosed herein and KRAS inhibitor are used in combination with a DNA-damaging treatment. The DNA-damaging treatment can be radiotherapy, or chemotherapy with a DNA-damaging antitumoral agent, or a combination thereof. DNA-damaging treatment refers to a treatment inducing DNA strand breakage, preferably relatively specifically in cancer cells.

DNA strand breakage can be achieved by ionized radiation (radiotherapy). Radiotherapy includes, but is not limited to, γ-rays, X-rays, and/or the directed delivery of radioisotopes to tumor cells. Other radiotherapies include microwaves and UV-irradiation. Other approaches to radiation therapy are also contemplated in the present invention.

DNA strand breakage can be achieved by radioisotope therapy, in particular by administration of a radioisotope, preferably a targeted radioisotope. Targeting can be due to the chemical properties of the isotope such as radioiodine which is specifically absorbed by the thyroid gland a thousand fold better than other organs. Alternatively, the targeting can be achieved by attaching to the radioisotope another molecule having targeting properties such hapten or antibody. Any of a number of suitable radioactive isotopes can be used, including, but not limited to, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-105, Palladium-109, Praseodymium-143, Promethium-149, Erbium-169, Iridium-194, Gold-198, Gold-199, and Lead-211.

The DNA-damaging antitumor agent is preferably selected from the group consisting of an inhibitor of topoisomerases I or II, a DNA crosslinker, a DNA alkylating agent, an anti-metabolic agent and inhibitors of the mitotic spindles.

Inhibitors of topoisomerases I and/or II include, but are not limited to, etoposide, topotecan, camptothecin, irinotecan, amsacrine, intoplicine, anthracyclines such as doxorubicine, epirubicine, daunorubicine, idanrubicine and mitoxantrone. Inhibitors of Topoisomerase I and II include, but are not limited to, intoplecin.

DNA crosslinkers include, but are not limited to, cisplatin, carboplatin and oxaliplatin.

Anti-metabolic agents block the enzymes responsible for nucleic acid synthesis or become incorporated into DNA, which produces an incorrect genetic code and leads to apoptosis. Non-exhaustive examples thereof include, without limitation, folic acid antagonists, pyrimidine analogs, purine analogs and adenosine deaminase inhibitors, and more particularly Methotrexate, Floxuridine, Cytarabine, 6-Mercaptopurine, 6-Thioguanine, Fludarabine phosphate, Pentostatine, 5-fluorouracil, gemcitabine and capecitabine.

The DNA-damaging anti-tumoral agent can be alkylating agents including, without limitation, nitrogen mustards, ethylenimine derivatives, alkyl sulfonates, nitrosoureas, metal salts and triazenes. Non-exhaustive examples thereof include Uracil mustard, Chlormethine, Cyclophosphamide (CYTOXAN®), Ifosfamide, Melphalan, Chlorambucil, Pipobroman, Triethylenemelamine, Triethylenethiophosphoramine, Busulfan, Carmustine, Lomustine, Fotemustine, cisplatin, carboplatin, oxaliplatin, thiotepa, Streptozocin, Dacarbazine, and Temozolomide.

Inhibitors of the mitotic spindles include, but are not limited to, paclitaxel, docetaxel, vinorelbine, larotaxel (also called XRP9881; Sanofi-Aventis), XRP6258 (Sanofi-Aventis), BMS-184476 (Bristol-Meyer-Squibb), BMS-188797 (Bristol-Meyer-Squibb), BMS-275183 (Bristol-Meyer-Squibb), ortataxel (also called IDN 5109, BAY 59-8862 or SB-T-101131; Bristol-Meyer-Squibb), RPR 109881A (Bristol-Meyer-Squibb), RPR 116258 (Bristol-Meyer-Squibb), NBT-287 (TAPESTRY), PG-paclitaxel (also called CT-2103, PPX, paclitaxel poliglumex, paclitaxel polyglutamate or Xyotax™), ABRAXANE® (also called Nab-Paclitaxel; ABRAXIS BIOSCIENCE), Tesetaxel (also called DJ-927), IDN 5390 (INDENA), Taxoprexin (also called docosahexanoic acid-paclitaxel; PROTARGA), DHA-paclitaxel (also called Taxoprexin®), and MAC-321 (WYETH). Also see the review of Hennenfent & Govindan (2006, Annals of Oncology, 17, 735-749).

Optionally, indirect KRAS therapeutic strategies towards KRAS-driven cancer can be used in combination with the use of a direct KRAS inhibitor and the Dbait according the invention.

The additional KRAS therapy or KRAS signaling therapy can target the same KRAS mutant pathway targeted by the Dbait and KRAS inhibitor according the invention. Alternatively, the additional KRAS therapy or KRAS signaling therapy can target different KRAS mutant pathways targeted by the Dbait and KRAS inhibitor according the invention.

### Cancers or Tumors to be Treated

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma.

Various cancers are also encompassed by the scope of the invention, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testis, genitourinary tract, urinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma (including cutaneous or peripheral T-cell lymphoma), Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, biliary tract cancer, endometrial cancer, uterine cancer, peritoneal cancer, kidney cancer, prostate cancer, thyroid cancer, neuroblastoma, pancreatic cancer, glioblastoma multiforme, cervical cancer, stomach cancer, bladder cancer, hepatocarcinoma, breast cancer, colon carcinoma, and head and neck cancer, retinoblastoma, gastric cancer, germ cell tumor, bone cancer, bone tumors, adult malignant fibrous histiocytoma of bone; childhood malignant fibrous histiocytoma of bone, sarcoma, pediatric sarcoma; myelodysplastic syndromes; neuroblastoma; testicular germ cell tumor, intraocular melanoma, myelodysplastic syndromes; myelodysplastic/myeloproliferative diseases, synovial sarcoma.

In one aspect, the cancer can be selected from the group consisting of lung, leukemia, lymphoma, sarcoma, melanoma, and cancers of the head and neck, kidney, ovary, pancreas, prostate, thyroid, lung, esophagus, breast, biliary tract malignancies, endometrial, cervical, bladder, brain, colorectum, liver, and cervix cancers.

In a particular aspect, the cancer is selected from the group consisting of lung cancer, in particular non-small cell lung cancer (NSCLC), leukemia, in particular acute myeloid leukemia, chronic lymphocytic leukemia, lymphoma, in particular peripheral T-cell lymphoma, chronic myelogenous leukemia, squamous cell carcinoma of the head and neck, advanced melanoma with BRAF mutation, colorectal cancer, gastrointestinal stromal tumor, breast cancer, in particular HER2⁺ breast cancer, thyroid cancer, in particular advanced medullary thyroid cancer, kidney cancer, in particular renal cell carcinoma, prostate cancer, glioma, pancreatic cancer, in particular pancreatic neuroendocrine cancer or pancreatic ductal adenocarninoma (PDAC), colon cancer, biliary tract malignancies, endometrial cancer, cervical cancer, bladder cancer and liver cancer, in particular hepatocellular carcinoma.

In one aspect, the cancer is driven by a KRAS mutation, in particular KRAS-4B isoform mutation. The mutation is selected from KRAS^{G12C}, KRAS^{G12V}, KRAS^{G12S}, KRAS^{G12D}, KRAS^{G13C} or KRAS^{G13D}. In a more particular aspect, the mutation is KRAS^{G12C} or KRAS^{G12D} mutation.

For instance, the cancer may be sarcoma and osteosarcoma such as Kaposi sarcome, AIDS-related Kaposi sarcoma, melanoma, in particular uveal melanoma, and cancers of the head and neck, kidney, ovary, pancreas, prostate, thyroid, lung, esophagus, breast in particular TNBC, bladder, small intestine, colorectum, liver, biliary tract, uterine, appendix, and cervix, testicular, gastrointestinal, urinary and endometrial and peritoneal cancers.

Preferably, the cancer may be pancreas, stomach, colon, small intestine, biliary tract, lung, endometrium, cervix, urinary tract, multiple myeloma, sarcoma, skin (melanoma), in particular uveal melanoma, and cancers of the head and neck, kidney, ovary, pancreas, prostate, thyroid, lung, esophagus, breast in particular TNBC, bladder, colorectum, liver, cervix, uterine corpus, endometrial and peritoneal cancer.

The cancer can be carcinoma or adenocarcinoma, such as lung adenocarcinoma, colon adenocarcinoma, non-small cell lung carcinoma (NSCLC), and colorectal adenocarcinoma, rectal carcinoma, pancreatic ductal adenocarcinoma and breast invasive ductal carcinoma.

In a preferred embodiment of the present invention, the cancer is a solid tumor. In one aspect, when the KRAS inhibitor is selected from the group consisting of AMG-510/Sotorasib (Amgen), MRTX-849/Adagrasib (Mirati Therapeutics), ARS-3248/JNJ-74699157 (Johnson & Johnson/Wellspring Biosciences), Compound B (Sanofi/X-Chem Pharmaceuticals), LY3499446 (Eli Lilly), ARS-853, ARS-1620, BI-2852, BI-1701963 (Boehringer Ingelheim), mRNA-5671 (Moderna Therapeutics), G12D inhibitor (Mirati), RAS(On)inhibitors (Revolution medicines), or BBP-454 (BridgeBio Pharma), and the cancer to be treated is driven by KRAS^{G12C} mutation.

For instance, when the mutation is KRAS^{G12C}, the cancer to be treated is preferably selected from lung cancer and lung adenocarcinoma, in particular non-small cell lung cancer, colorectal cancer and colon adenocarcinoma, in particular metastatic or advanced colorectal cancer, pancreatic cancer, breast cancer, in particular early breast cancer and TNBC, thyroid cancer, in particular medullary thyroid cancer, squamous cell carcinoma of the head and neck and glioma.

For instance, when the mutation is KRAS^{G12V}, the cancer to be treated is preferably selected from pancreatic adenocarcinoma, lung adenocarcinoma, colon adenocarcinoma, colorectal adenocarcinoma, and rectal adenocarcinoma.

For instance, when the mutation is KRAS^{G12S}, the cancer to be treated is preferably selected from colon adenocarcinoma, lung adenocarcinoma, colorectal adenocarcinoma, rectal adenocarcinoma, and breast invasive ductal carcinoma.

For instance, when the mutation is KRAS^{G12D}, the cancer to be treated is preferably selected from pancreatic adenocarcinoma, colon adenocarcinoma, lung adenocarcinoma, colorectal adenocarcinoma, and rectal adenocarcinoma.

For instance, when the mutation is KRAS^{G13C}, the cancer to be treated is preferably selected from lung adenocarcinoma and colon adenocarcinoma.

For instance, when the mutation is KRAS^{G13D}, the cancer to be treated is preferably selected from colon adenocarcinoma, colorectal adenocarcinoma, lung adenocarcinoma, rectal adenocarcinoma, and endometrial adenocarcinoma.

In one aspect, when the KRAS inhibitor is Pan-KRAS Inhibitor selected from BI 1701963 or onvansertib, the cancer to be treated is driven by KRAS^{G12C}, KRAS^{G12V}, KRAS^{G12S}, KRAS^{G12D}, KRAS^{G13C} or KRAS^{G13D} mutation.

The pharmaceutical compositions and the products, kits, combinations or combined preparations described in the invention may be useful for inhibiting the growth of solid tumors, decreasing the tumor volume, preventing the metastatic spread of tumors and the growth or development of micrometastases, preventing the tumor recurrence and preventing the tumor relapse. The pharmaceutical compositions and the products, kits, combinations, or combined preparations described in the invention are in particular suitable for the treatment of poor prognosis patients or of radio- or chemo-resistant tumors. In a particular embodiment, the cancer is a high-grade or advanced cancer or is a metastatic cancer.

### Regimen, Dosages and Administration Routes

The effective dosage of each of the combination partners employed in the combined preparation of the invention may vary depending on the particular compound or pharmaceutical composition employed, the mode of administration, the condition being treated, the severity of the condition being treated. Thus, the dosage regimen of the combined preparation of the invention is selected in accordance with a variety of factors including the route of administration and the patient status. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The pharmacological activity of a combination of the invention may, for example, be demonstrated in a clinical study or more preferably in a test procedure. Suitable clinical studies are, for example, open label non-randomized, dose escalation studies in patients with advanced tumors. Such studies can prove the synergism of the active ingredients of the combination of the invention. The beneficial effects on proliferative diseases can be determined directly through the results of these studies or by changes in the study design which are known as such to a person skilled in the art. Such studies are, in particular, suitable to compare the effects of a monotherapy using the active ingredients and a combination of the invention. Preferably, the combination partner (a) is administered with a fixed dose and the dose of the combination partner (b) is escalated until the maximum tolerated dosage is reached. Alternatively, the combination partner (b) is administered with a fixed dose and the dose of the combination partner (a) is escalated until the maximum tolerated dosage is reached.

In some embodiments, “combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Preferably, the Dbait molecule and the KRAS inhibitor are administered concomitantly or simultaneously.

The term “concomitantly” is used herein to refer to administration of two or more therapeutic agents, give in close enough temporal proximity where their individual therapeutic effects overlap in time. Accordingly, concurrent administration includes a dosing regimen when the administration of one or more agent(s) continues after discontinuing the administration of one or more other agent(s).

The Dbait molecule and the KRAS inhibitor can have same or different administration regimen. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, or any combination thereof. For example, in one embodiment, the first agent can be administered prior to the second therapeutic agent, for e.g. 1 week. In another, the first agent can be administered prior to (for example 1 day prior) and then concomitant with the second therapeutic agent.

The Dbait molecule and the KRAS inhibitor may be administered by the same route or by distinct routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Therapeutic agents may also be administered in alternation. The administration route could be oral, parenteral, intravenous, intratumoral, subcutaneous, intracranial, intraartery, topical, rectal, transdermal, intradermal, nasal, intramuscular, intraosseous, and the like.

The treatment may include one or several cycles, for instance two to ten cycles, in particular two, three, four or five cycles. The cycles may be continued or separated. For instance, each cycle is separated by a period of time of one to eight weeks, preferably three to four weeks.

Further aspects and advantages of the present invention will be described in the following examples, which should be regarded as illustrative and not limiting.

### EXAMPLES

The Inventors of the present invention have identified and demonstrated in vitro for the first time that KRAS inhibitor is associated with the occurrence of persister cancer cells during a treatment of cancer with this KRAS inhibitor.

### Example 1

### Material and Methods

### Drugs

Different drugs were used throughout experiments. KRAS^{G12C} specific inhibitors, AMG-510 and MRTX-849, were purchased from Selleckchem and diluted on dimethyl sulfoxide (DMSO) to a stock concentration of 10 mM and of 1 mM, respectively. They are summarized in Table 1 below.

TABLE 1
Drugs used to treat cells with their function, the supplier, the solvent
used and their final concentration.

| | | | | Final con- |
|---|---|---|---|---|
| Name | Function | Supplier | Solvent | centration |
| AMG-510 | KRASG12Ci | Selleckchem | Dimethyl sulfoxide | 10 mM |
| | | | (DMSO) | |
| MRTX849 | KRASG12Ci | Selleckchem | Dimethyl sulfoxide | 1 mM |
| | | | (DMSO) | |

AsiDNA was manufactured by Avecia (USA) and diluted on purified water to a stock concentration of 943 μM.

### Cell Culture

Cell cultures were performed with non-small cell lung (NSCLC) cancer cell line NCI-H23, a KRAS^{G12C} mutant cell line (heterozygous mutation). The NCI-H23 cell line was purchased from the ATCC. Cells were grown according to the supplier's instructions and maintained at 37° C. in a humidified atmosphere at 5% CO2. Medium was renewed twice a week and cells were passed when the confluence reached 70-80% depending on cell lines. Each cell line was generally kept in passage for no more than 2 months.

### Selection of Resistant Populations

Cells were seeded in T75 flasks with 5.10⁵ cells per flask or in 25 cm² culture dishes with 10⁵ cells per dish and incubated 24 hours at 37° C. before addition of KRAS^{G12C}i (AMG-510 20 μM or 1 μM MRTX-849) with or without AsiDNA (doses ranging from 500 nM to 2500 nM). AsiDNA was added either concomitantly and continuously with the KRAS^{G12C} inhibitor (AMG-510 or MRTX-849) or two weeks from the KRAS^{G12C}i treatment start. At least 3 to 6 independent populations were treated for each condition. Drugs were renewed twice a week to maintain a high pressure of resistance selection. Cells were harvested, washed and counted approximately ones a week after staining with 0.4% trypan blue (Sigma Aldrich, Saint-Louis, USA) using an automated cell counter (EVE™-Nanoentek).

### βgal Staining

Slow cycling/senescent drug-tolerant cells were identified using Senescence detection kit according to the manufacturer instructions (Abcam; ab65351). Briefly, cells were seeded in 12-well culture plates and incubated overnight at 37° C. Cells were then washed twice with PBS, fixed during 10 minutes with 0.5 ml of Fixative solution at room temperature, washed twice with PBS and then stained using 0.5 ml of the Staining solution mix overnight at 37° C. Cells are then analyzed under microscope for development of blue color.

### Flow Cytometry

NCI-H23 were seeded in T25 flasks at 4.105 cells per flask and then treated for 24 h with AsiDNA™ at 100 nM or 5 μM for 24 hours. For intracellular staining (pPERK and CDK p16/CDK p21, mTOR, Bcl-2 staining), cells were washed, then fixed in PBS/70% Ethanol during at least 1 hour at 4° C. Cells were then washed, permeabilized with PBS/0.2% TritonX-100 solution at RT for 30 min, and saturated with PBS/2% Bovine Serum Albumin (BSA) solution at RT for 10 min. Then, cells were washed with PBS and incubated 1 hour with respectively, FITC-conjugated anti pPERK (Biorbyt, UK, 1:120), Alexa647-conjugated anti-CDK p16 (Cell signaling, Danvers Mass., USA, 1:50), Alexa488-conjugated anti-CDK p21 (Cell signaling, Danvers Mass., USA, 1:50), Alexa488-conjugated anti-mTOR (Cell signaling, Danvers Mass., USA, 1:50), Alexa647-conjugated anti-Bcl-2 (Cell signaling, Danvers Mass., USA, 1:50) before flow cytometry analysis (Guava EasyCyte 12H, Luminex, Germany). For cell-surface receptors staining with a PE-conjugated anti-transferrin receptor antibody (Thermofisher, Waltham Mass., USA, 1:20). Stained cells were then washed with PBS and fluorescence intensities were acquired with a Guava EasyCyte 12H flow cytometer (Luminex, Germany). Data were analyzed using FlowJo software (Tree Star, CA, USA), cells were harvested and washed directly after treatment end, and then incubated for 1 hour at 4° C.

### Results

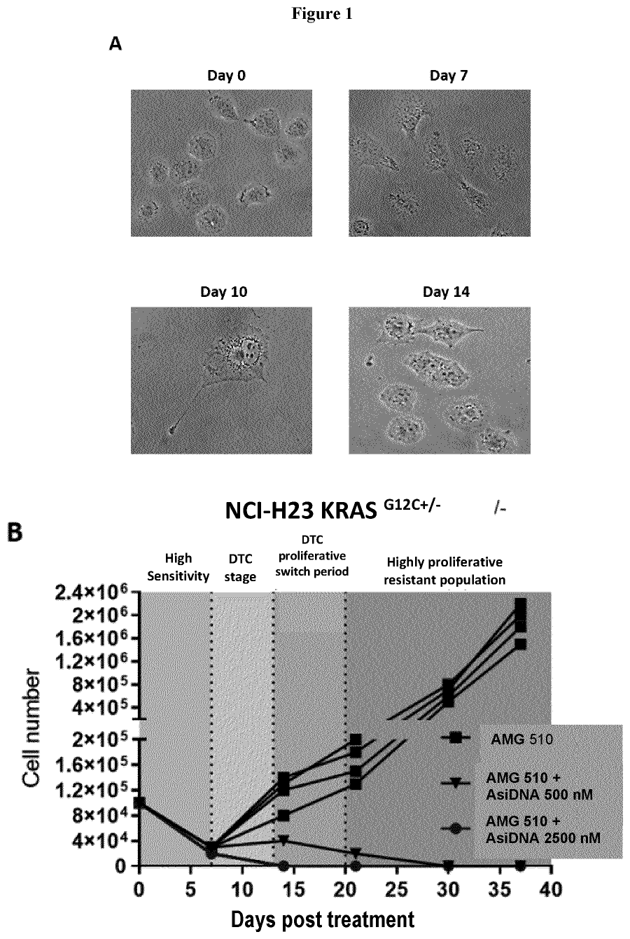

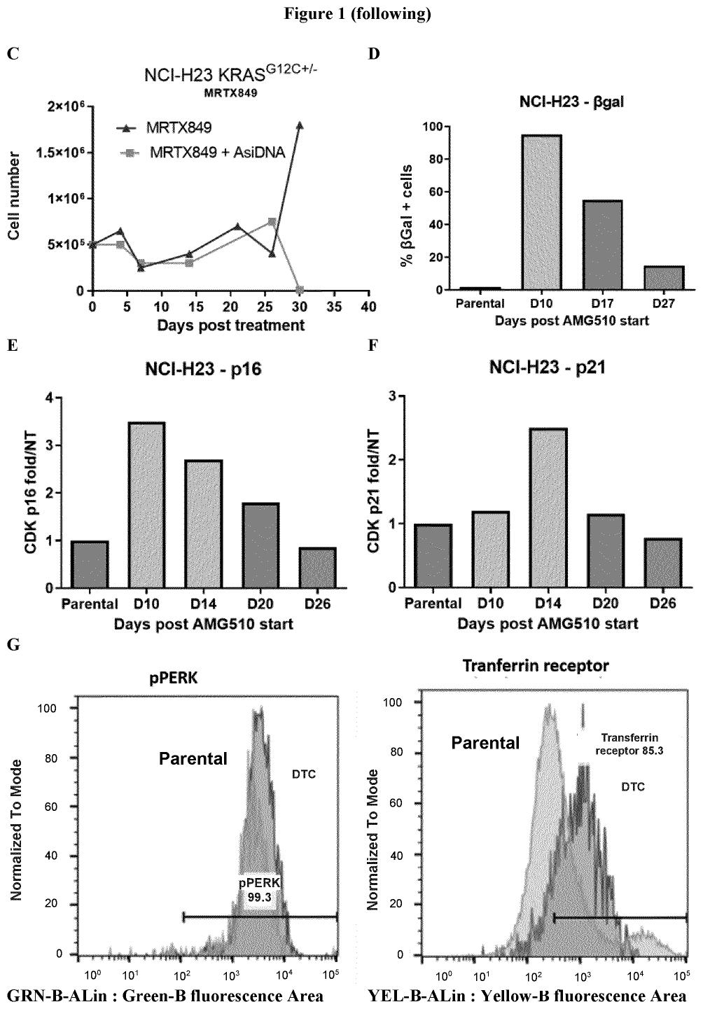

In order to select resistance to the KRAS^{G12C} inhibitor AMG-510 and MRTX-849 in vitro, the inventors performed continuous treatment at a high dose/IC₉₀ doses (corresponding to approximately 90% of inhibition of cells growth) on the KRAS^{G12C+/-} mutant NSCLC cancer cell line NCI-H23 (FIG. 1). Each population per condition was considered all the time as independent. Cells were initially very sensitive to these inhibitors (AMG-510 or MRTX-849), with only few residual cells remaining in culture dishes during the two first weeks of treatment (FIGS. 1 B and C). These residual cells were phenotypically very different from parental NCI-H23 cells, with senescence-associated phenotypic hallmarks including a non-proliferative profile and cell enlargement (FIG. 1A—Day 10), β-gal positive staining (FIG. 1D), and p16 and p21 expression increases (FIGS. 1E and F). Interestingly, this “senescence-like” phenotype was only transient, since cells lost this larger shape (Day 14), the β-gal positive staining decreased (90% of the NCI-H23 cells were in a senescent state at day 10 of treatment, 50% at Day 17 and only 10% at Day 27), the higher p16 and p21 expression (at Day 10 and D14 respectively) declined. NCI-H23 cells re-started to proliferate starting from three to four weeks after treatment. Therefore, NCI-H23 cells became senescent during the first period of treatment, before leaving this cellular state. To check if these changes are associated to a proliferative switch of cells under treatment to generate resistance, cells were counted one to two times per week (FIGS. 1B and C). We noticed that starting from two to three weeks of AMG-510 treatment, cells restarted to divide and entered a proliferative state. Coherently, this regrowth appeared few days after the start of senescent markers decrease. All these changes were acquired under AMG-510 or MRTX-849 treatment, indicating de-novo resistance (FIG. 1B—Squares, FIG. 1C—Triangles), which evolved from senescent-like dormant persister cells with a DTC phenotype.

To confirm the implication of DTCs in resistance to KRAS^{G12C}i, inventors checked other DTCs specific biomarkers as the increase of endoplasmic reticulum (ER) stress (PERK/pPERK pathway) and ferroptosis (transferrin receptor 1) (FIG. 1G). NCI-H23 cells were continuously treated with AMG-510 (20 μM) to induce DTCs, and PERK/pPERK receptor and transferrin receptor 1 were analyzed in senescent cells compared to parental cells. Compared to parental cell line, senescent cells showed an increase of both pPERK and transferrin receptors (FIG. 1G) indicating that KRAS^{G12C}i induced persistence of drug tolerant cells.

In order to test the potential of AsiDNA to prevent resistance to KRAS^{G12C} inhibitor, the inventors used low doses of AsiDNA (500 and 2500 nM—sub-cytotoxic doses) in combination with AMG-510, or low dose of AsiDNA (2500 nM) in combination with MRTX-849 and quantified cell proliferation in comparison to monotherapies (AMG-510 or MRTX-849 alone treated cells). AsiDNA did not modified the cytotoxic effect of AMG-510 nor of MRTX-849 in the first days demonstrating that AsiDNA did not interfere with the efficacy of AMG-510 or of MRTX-849 on KRAS^{G12C} cancer cells. Interestingly, cells treated concomitantly and continuously with AMG-510 or MRTX-849+AsiDNA died and didn't restart proliferation after more than one month of treatment (FIGS. 1B—Circles and Triangles, and C—Squares), compared to cells treated with AMG-510 or MRTX-849 alone, which escaped dormancy and re-started rapid proliferation (FIGS. 1B—Squares, and C—Triangles).

Taken together, inventors demonstrated that NCI-H23 cancer cells entered in a “Drug-tolerant” state under KRAS^{G12C}i. Thus, these Drug-tolerant cells (DTCs) underwent a phenotypic and gene expression switch to become proliferative, and responsible of rapid KRAS^{G12C}i therapy resistance, a feature vulnerable to AsiDNA. Resistance to KRAS^{G12C}i may be abrogated by AsiDNA, triggering an irreversible senescence-like state followed by cell death.

### Example 2

### Material and Methods

### Drugs

KRAS^{G12C} specific inhibitor, AMG-510, was purchased from Selleckchem and diluted on dimethyl sulfoxide to a stock concentration of 10 mM. AsiDNA was manufactured by Avecia (USA) and diluted on purified water to a stock concentration of 943 μM.

### Cell Culture

Cell cultures were performed with NCI-H23, a KRAS^{G12C} mutant cell line (heterozygous mutation). NCI-H23 cell line was purchased from the ATCC. Cells were grown according to the supplier's instructions and maintained at 37° C. in a humidified atmosphere at 5% CO2. Medium was renewed twice a week and cells were passed when the confluence reached 70-80% depending on cell lines. Each cell line was generally kept in passage for no more than 2 months.

### Selection of Resistant Populations

Cells were seeded in in T75 flasks with 5.10⁵ cells per flask and incubated 24 hours at 37° C. before addition of AMG-510 (20 μM) with or without AsiDNA (doses ranging from 100 nM to 2500 nM). AsiDNA was added either concomitantly and continuously with the KRAS^{G12C} inhibitor or two weeks from the AMG-510 treatment start. At least 3 to 6 independent populations were treated for each condition. Drugs were renewed twice a week to maintain a high pressure of resistance selection. Cells were harvested, washed and counted approximately ones a week after staining with 0.4% trypan blue (Sigma Aldrich, Saint-Louis, USA) using an automated cell counter (EVE™-Nanoentek).

### Immunofluorescence Analysis

For immunostaining, cells are seeded on Lab-Tek® II Chamber Slide™ (Nunc, Rochester, USA) at 2.10⁴ cells/slide and incubated at 37° C. during 24 hours. Cells are then treated with 100 nM or 5 μM AsiDNA to analyze the amount of AsiDNA-induced false DNA damage. Twenty-four hours after treatment, cells are fixed for 20 minutes in 4% paraformaldehyde/PBS 1×, permeabilized in 0.5% Triton X-100 for 10 minutes, blocked with 2% BSA/PBS 1× for 15 min. All antibodies were fluorochrome-coupled. The following antibody was used: Alexa488-conjugated γH2AX (1:200) and incubated for 1 hour at room temperature. DNA was stained for 5 minutes with 6-diamidino-2-phenylindole (DAPI). Cells are then analyzed under microscope (Nikon Eclipse TS100, Nikon corp. Tokyo, Japan).

### Cell Survival Analysis (IC₅₀)

XTT is used to assess cell viability as a function of redox potential. Actively dividing cells convert the water-soluble XTT to a water-unsoluble, orange colored formazan product. Cells were seeded on a 96 well plate at 2.10³ cells per well and then treated for a week with AMG-510 at a starting concentration of 20 μM and AsiDNA™ at a starting concentration of 100 nM. Cell proliferation Kit II (XTT) (Roche, Basel, Switzerland) was used and XTT mix was applied during 6 hours before absorbance analysis in a microplate reader. (Enspire™ Perkin-Almer). Statistical and IC₅₀ analysis were calculated using GraphPad Prism software (GraphPad Prism 5, San Diego, Calif., USA).

### Results

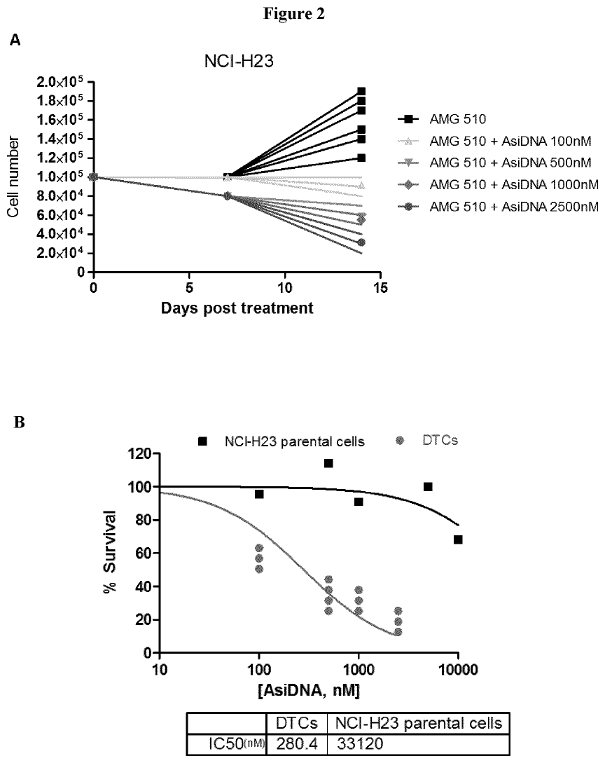

To confirm that AsiDNA inhibits resistance to AMG-510, the inventors performed other series of AMG-510 resistance selection with or without AsiDNA at lower doses (2500 nM to 100 nM). AsiDNA completely inhibited acquired resistance to AMG-510 by inhibiting proliferation from DTC stage even at the lowest dose of 100 nM (FIG. 2A—Light grey Triangles). To further verify whether DTCs are highly sensitive to AsiDNA, the inventors compared their sensitivity to those of parental cells day 14 after treatment start (FIG. 2B). As expected, NCI-H23 parental cells are not sensitive to AsiDNA at these doses, with an estimated IC₅₀ of approximately 33 μM compared to DTCs showing an IC₅₀ more than 100-fold lower (280 nM), supporting the idea that DTCs are highly sensitive to AsiDNA, probably due to different mechanisms (FIG. 2B).

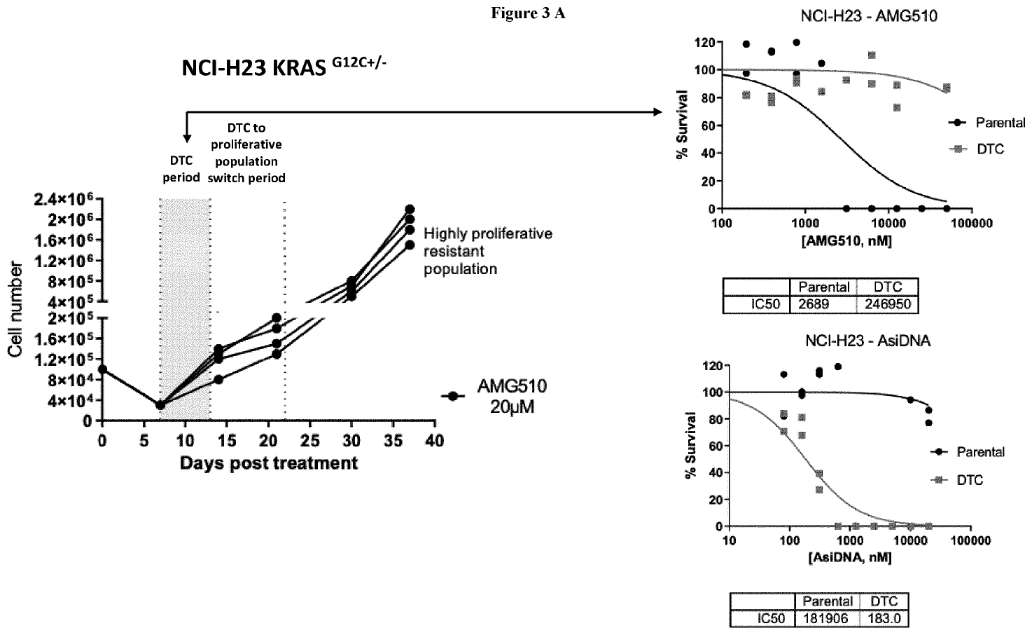

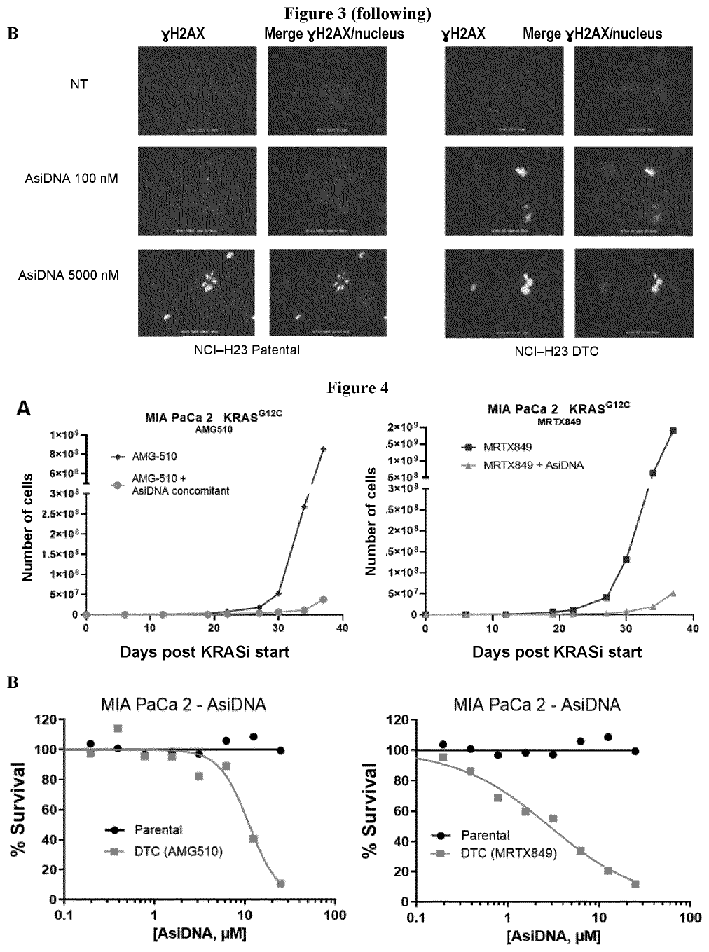

In another set of experiments, inventors evaluated if KRAS^{G12C}i-induced DTCs are highly sensitive to AsiDNA compared to parental cells (FIG. 3). Briefly, NCI-H23 cells were treated with AMG-510 for 10 days to select a population mostly constituted of DTCs, then AMG-510 treatment was arrested and DTCs were treated with increasing doses of AsiDNA or of AMG-510 alone. DTCs were very sensitive to AsiDNA, which abrogated proliferation and triggered DTCs death (FIG. 3A—lower right part), with an estimated IC₅₀ of 183 nM, 1000-fold lower compared to parental cells, supporting the idea that DTCs are highly sensitive to AsiDNA.

To decipher the mechanisms underlying the hypersensitivity of DTCs to AsiDNA, inventors analyzed AsiDNA-induced target engagement in DTCs compared to parental cells. Compared to parental NCI-H23 cells, where AsiDNA induced a pan-nuclear γH2AX starting from the dose of 5 μM, with no activity at 100 nM (FIG. 3B), DTCs showed a clear γH2AX staining even at the low dose of 100 nM (FIG. 3B), which could explain, at least in part, the hypersensitivity of AMG-510-induced DTCs to AsiDNA.

### Example 3

### Material and Methods

### Drugs

Different drugs were used throughout experiments. KRAS^{G12C} specific inhibitors, AMG-510 and MRTX-849, were purchased from Selleckchem and diluted on dimethyl sulfoxide (DMSO) to a stock concentration of 10 mM and of 1 mM, respectively. They are summarized in Table 1 above of Example 1.

AsiDNA was manufactured by Avecia (USA) and diluted on purified water to a stock concentration of 943 μM.

### Cell Culture

Cell cultures were performed with a pancreatic cancer cell line MIA PaCa-2. The MIA PaCa-2 cell lines were purchased from the ATCC. Cells were grown according to the supplier's instructions and maintained at 37° C. in a humidified atmosphere at 5% CO2. Medium was renewed twice a week and cells were passed when the confluence reached 70-80% depending on cell lines. Each cell line was generally kept in passage for no more than 2 months.

### Selection of Resistant Populations

Cells were seeded in T75 flasks with 5.10⁵ cells per flask and incubated 24 hours at 37° C. before addition of KRAS^{G12C}i (1 μM AMG-510 or 1 μM MRTX-849) with or without AsiDNA (5000 nM). AsiDNA was added either concomitantly and continuously with the KRAS^{G12C} inhibitor (AMG-510 or MRTX-849) or two weeks from the KRAS^{G12C}i treatment start. At least 3 to 6 independent populations were treated for each condition. Drugs were renewed twice a week to maintain a high pressure of resistance selection. Cells were harvested, washed and counted approximately ones a week after staining with 0.4% trypan blue (Sigma Aldrich, Saint-Louis, USA) using an automated cell counter (EVE™-Nanoentek).

### Cell Survival Analysis (IC₅₀)

XTT is used to assess cell viability as a function of redox potential. Actively dividing cells convert the water-soluble XTT to a water-unsoluble, orange colored formazan product. Cells are seeded on a 96 well plate at 2.10³ cells per well and then treated for a week with AsiDNA at a starting concentration of 20 or 50 μM. Cell proliferation Kit II (XTT) (Roche, Basel, Switzerland) was used and XTT mix was applied during 6 hours before absorbance analysis in a microplate reader. (Enspire™ Perkin-Almer). Statistical and IC₅₀ analysis were calculated using GraphPad Prism software (GraphPad Prism 5, San Diego, Calif., USA).

### Results

In addition to lung cancer, KRAS^{G12C}i are also highly relevant for the treatment of several other cancers KRAS^{G12C}-dependent like pancreatic cancer. To check if resistance to KRAS^{G12C}i in pancreatic cancer is also DTCs related, inventors treated the KRAS^{G12C}-mutated MIA PaCa-2 pancreatic cancer model continuously with AMG-510 (1 μM) or MRTX-849 (1 μM) (FIG. 4A). As in lung cancer cells, continuous treatment with AMG-510 or MRTX-849 induced the emergence of acquired resistance, approximately at 1 month after treatment start (FIG. 4A—Black curves). In fact, DTCs phenotypic switch to a proliferative state occurred approximately 30 to 35 days after KRAS^{G12C}i treatment start (FIG. 4A). To confirm that AsiDNA could also abrogate resistance to KRAS^{G12C}i in pancreatic cancer, inventors tested the efficacy of the concomitant combined treatment KRAS^{G12C}i+AsiDNA in resistance prevention. Concomitant treatment with AsiDNA and KRAS^{G12C}i rapidly and completely inhibited acquired resistance emergence (FIG. 4A—Grey curves). Inventors also evaluated if KRAS^{G12C}i-induced DTCs are highly sensitive to AsiDNA compared to parental cells in pancreatic cancer (FIG. 4B). Briefly, MIA PaCa-2 cells were treated with KRAS^{G12C}i for 20 days to select a population mostly constituted of DTCs, then KRAS^{G12C}i treatment was arrested and DTCs were treated with increasing doses of AsiDNA. DTCs were highly sensitive to AsiDNA (FIG. 4B—Grey curves) compared to parental MIA PaCa-2 cells (FIG. 4B—Black curves).

These results suggest that the DTCs-induced KRAS^{G12C}i resistance is common at least to lung and pancreatic cancer cells and can be similarly targeted by AsiDNA. In addition, these results highlight the potential of AsiDNA to abrogate resistance to all different KRAS^{G12C}i.

1-16. (canceled)

17. A pharmaceutical composition comprising a Dbait molecule and a protein KRAS inhibitor, wherein the Dbait molecule has one of the following formulae: 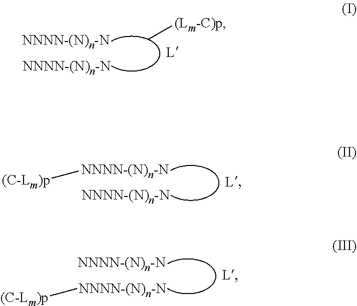
wherein N is a deoxynucleotide, n is an integer from 15 to 195, the underlined N refers to a nucleotide having or not a modified phosphodiester backbone, L′ is a linker, C is the molecule facilitating endocytosis selected from a lipophilic molecule or a ligand which targets cell receptor enabling receptor mediated endocytosis, L is a linker, m and p, independently, are an integer being 0 or 1.

18. The pharmaceutical composition according to claim 17, wherein the Dbait molecule has the following formula:

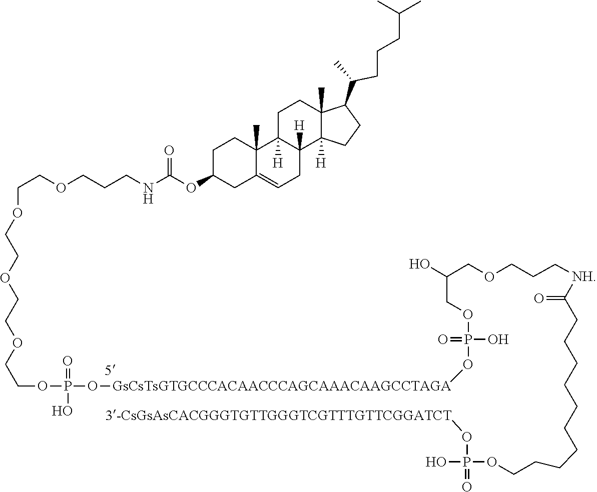

19. The pharmaceutical composition according to claim 17, wherein the KRAS inhibitor is a direct KRAS inhibitor selected from the group consisting of specific covalent KRAS inhibitors and multivalent small-molecule pan KRAS inhibitors.

20. The pharmaceutical composition according to claim 19, wherein the KRAS inhibitor is selected from the group consisting of AMG-510/sotorasib (Amgen/Carmot Therapeutics), MRTX-849/Adagrasib (Mirati Therapeutics), ARS-3248/JNJ-74699157 (Johnson & Johnson/Wellspring Biosciences), Compound B (Sanofi/X-Chem Pharmaceuticals), LY3499446 (Eli Lilly), ARS-853, ARS-1620, BI-2852, BI-1701963 (Boehringer Ingelheim), mRNA-5671 (Moderna Therapeutics), G12D inhibitor (Mirati), RAS(On)inhibitors (Revolution medicines), and BBP-454 (BridgeBio Pharma).

21. The pharmaceutical composition according to claim 19, wherein the KRAS inhibitor is a KRASG12C inhibitor directly targeting and binding mutant KRASG12C protein.

22. The pharmaceutical composition according to claim 17, wherein the KRAS inhibitor leaves wild-type KRAS protein untouched.

23. A combination comprising a Dbait molecule and a protein KRAS inhibitor, wherein the Dbait molecule has one of the following formulae: 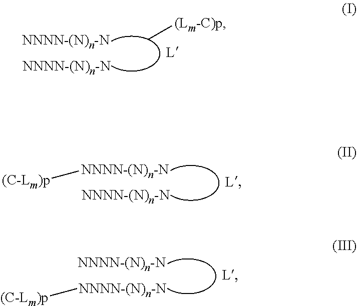
wherein N is a deoxynucleotide, n is an integer from 15 to 195, the underlined N refers to a nucleotide having or not a modified phosphodiester backbone, L′ is a linker, C is the molecule facilitating endocytosis selected from a lipophilic molecule or a ligand which targets cell receptor enabling receptor mediated endocytosis, L is a linker, m and p, independently, are an integer being 0 or 1.

24. The combination according to claim 23, wherein the Dbait molecule has the following formula:

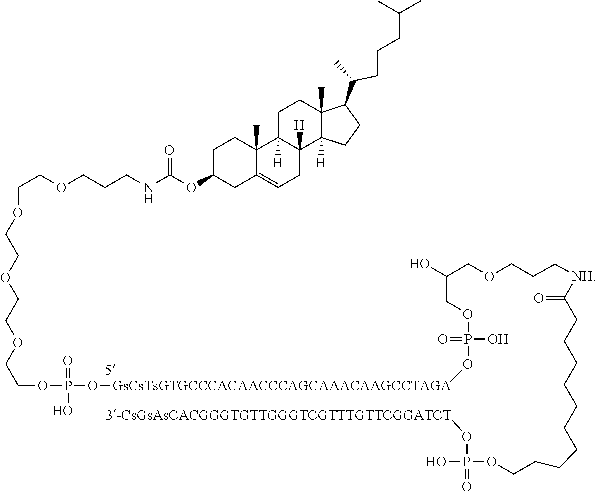

25. The combination according to claim 23, wherein the KRAS inhibitor is a direct KRAS inhibitor selected from the group consisting of specific covalent KRAS inhibitors and multivalent small-molecule pan KRAS inhibitors.

26. The combination according to claim 25, wherein the KRAS inhibitor is selected from the group consisting of AMG-510/sotorasib (Amgen/Carmot Therapeutics), MRTX-849/Adagrasib (Mirati Therapeutics), ARS-3248/JNJ-74699157 (Johnson & Johnson/Wellspring Biosciences), Compound B (Sanofi/X-Chem Pharmaceuticals), LY3499446 (Eli Lilly), ARS-853, ARS-1620, BI-2852, BI-1701963 (Boehringer Ingelheim), mRNA-5671 (Moderna Therapeutics), G12D inhibitor (Mirati), RAS(On)inhibitors (Revolution medicines), and BBP-454 (BridgeBio Pharma).

27. The combination according to claim 25, wherein the KRAS inhibitor is a KRASG12C inhibitor directly targeting and binding mutant KRASG12C protein.

28. The combination according to claim 25, wherein the KRAS inhibitor leaves wild-type KRAS protein untouched.

29. A method of treating cancer comprising administering a pharmaceutical composition according to claim 23 to a subject in need of treatment.

30. The method according to claim 29, wherein the cancer is a cancer driven by a KRAS mutation selected from the group consisting of KRASG12C, KRASG12V, KRASG12S, KRASG12D, KRASG13C, KRASG13D, KRASG12C, and KRASG12D.

31. The method according to claim 29, wherein the cancer is selected from the group consisting of a cancer of head and neck, pancreas, stomach, colon, colorectum, small intestine, biliary tract, kidney, ovary, prostate, thyroid, esophagus, breast, bladder, lung, liver, uterine corpus, endometrium, cervix, urinary tract, peritoneal cancers, multiple myeloma, sarcoma, skin cancer, melanoma, uveal melanoma, and hematopoietic cancers.

32. A method of treating cancer comprising administering a therapeutically effective amount of a combination according to claim 23 to a subject in need of treatment.

33. The method according to claim 32, wherein the cancer is a cancer driven by a KRAS mutation selected from the group consisting of KRASG12C, KRASG12V, KRASG12S, KRASG12D, KRASG13C, KRASG13D, KRASG12C, and KRASG12D.

34. The method according to claim 32, wherein the cancer is selected from the group consisting of a cancer of head and neck, pancreas, stomach, colon, colorectum, small intestine, biliary tract, kidney, ovary, prostate, thyroid, esophagus, breast, bladder, lung, liver, uterine corpus, endometrium, cervix, urinary tract, peritoneal cancers, multiple myeloma, sarcoma, skin cancer, melanoma, uveal melanoma, and hematopoietic cancers.

35. A method of delaying the development of a cancer resistant to a KRAS inhibitor in a patient comprising administering a composition according to claim 17 to a patient in need of treatment.

36. A method of delaying the development of a cancer resistant to a KRAS inhibitor in a patient comprising administering a combination according to claim 23 to a patient in need of treatment.

37. A method of treating cancer persister cells is a subject having cancer comprising administering a composition according to claim 17 to said subject.

38. A method of treating cancer persister cells is a subject having cancer comprising administering a combination according to claim 23 to said subject.


SEQUENCE LISTING (6 sequences)

SEQ ID NO: 1 - DNA, 32 bp, artificial sequence
  Dbait32
acgcacgggtgttgggtcgtttgttcggatct

SEQ ID NO: 2 - DNA, 32 bp, artificial sequence
  Dbait32Ha
cgtaggtctgtttggtggctttgcagtggcac

SEQ ID NO: 3 - DNA, 32 bp, artificial sequence
  Dbait32Hb
gctaggcttgtttgctgggttgtaggcacagc

SEQ ID NO: 4 - DNA, 32 bp, artificial sequence
  Dbait32Hc
gctgtgcccacaacccagcaaacaagcctaga

SEQ ID NO: 5 - DNA, 32 bp, artificial sequence
  Dbait32Hd
gctaggtctgtttggtggctttgcagtggcac

SEQ ID NO: 6 - DNA, 32 bp, artificial sequence; identical to SEQ ID NO: 4
  Dbait molecule IIa
  misc_feature: at the 3′ end of the complementary strand the three last nucleotides with phosphorothioate or methylphosphonate backbone
  misc_feature (1)..(1): at the 5′ end, Lm = carboxamido oligoethylene glycol + C = single or double chain fatty acids, cholesterol, sugars, peptides or proteins
  misc_feature (1)..(1): at the 5′ end, Lm = tetraethyleneglycol + C = cholesterol
  stem_loop (1)..(32)
  modified_base (1)..(3): mod_base= phosphorothioate or methylphosphonate backbone
  misc_feature (32)..(32): loop L′ = 1,19-bis(phospho)-8-hydraza-2-hydroxy-4-oxa-9-oxo-nonadecane